(19) United States
(12) Patent Application Publication
(10) Pub. No.: US 2014/0277501 A1
(43) Pub. Date: Sep. 18, 2014

(54) EXPANDING INTERBODY VERTEBRAL IMPLANT

(71) Applicant: Aurora Spine, Inc., Carlsbad, CA (US)

(72) Inventors: Trent James Northcutt, Oceanside, CA (US); Laszlo Garamszegi, Mission Viejo, CA (US)

(21) Appl. No.: 14/210,392

(22) Filed: Mar. 13, 2014

Related U.S. Application Data
(60) Provisional application No. 61/781,013, filed on Mar. 14, 2013.

(51) Int. Cl. A61F 2/44

(52) U.S. Cl. CPC A61F 2/447
USPC 623/17.16

(57) ABSTRACT

A height-adjustable vertebral spacer is described. The spacer has a superior member and an inferior member. The superior member has a superior vertebral interface and an inferior nesting interface. The superior vertebral interface includes angled teeth, and the inferior nesting interface has one or more lateral walls with one or more rows of slits formed therein. The inferior member has an inferior vertebral interface and one or more lateral walls with one or more rows of ridges protruding inwardly from the one or more lateral walls. Each of the ridges has an angled bottom side and a flat upper side that is perpendicular to the lateral wall from which it protrudes. When the superior member is mated with the inferior member the one or more ridges of the inferior member are oriented to mate with the one or more slits of the superior member.

The present invention claims priority from U.S. Provisional Application Ser. No. 61/781,013, filed Mar. 14, 2013, the entirety of which is incorporated herein by reference.

### BACKGROUND

The invention relates to the restoration of intervertebral disc space, and vertebral stabilization for spinal fusion.

The spinal column is a physical structure that contains mostly ligaments, muscles, vertebrae and intervertebral discs. In human anatomy, the spinal column (also called vertebral column) consists of 24 articulating vertebrae, and nine fused vertebrae in the sacrum and the coccyx. It is situated in the dorsal aspect of the torso, separated by intervertebral discs. It houses and protects the spinal cord in its spinal canal, and hence is commonly called the spine, or simply backbone.

There are normally 33 vertebrae in humans, including the five that are fused to form the sacrum (the others are separated by intervertebral discs) and the four coccygeal bones that form the tailbone. The upper three regions comprise the remaining 24, and are grouped under the names cervical (seven vertebrae), thoracic (12 vertebrae) and lumbar (five vertebrae), according to the regions they occupy.

Between each pair of vertebrae is a disk-shaped pad of fibrous cartilage with a jelly-like core, which is called the intervertebral disc. These discs cushion the vertebrae during movement. The thickness or height of the disc determines and fixes the distance between two successive vertebrae.

Disease or damage to the discs can cause pain and suffering that can be temporary or constant and permanent. Many different diseases or traumatic events can cause damage to a disc that is irreversible. When that happens, one of the remedies is to remove the disc and fuse the two vertebrae that were separated by the disc. This is called a spinal fusion procedure, also known as spondylodesis or spondylosyndesis. Supplementary bone tissue, either from the patient (autograft), a donor (allograft), or synthetic bone substitute, is used in conjunction with the body's natural bone growth (osteoblastic) processes to fuse the vertebrae.

The fusion is accomplished by removing the disk, creating a space between the vertebrae, and fusing the two vertebrae.

Over the years, a variety of vertebral spacers or cages have been developed that replace the discs and maintain space between successive vertebrae while the vertebrae fuse over time. These spacers can be temporary, but usually they are permanently implanted. They are sometimes called cages, because they have cavities or open spaces within them that can be packed with supplementary bone tissue to promote fusion between successive vertebrae.

The conventional cage spacers are typically cut subject to the height of the resected disc so that the spacer can be set in between the adjacent upper and lower vertebral bodies. The major drawback of this design of cage type vertebral spacer is the non-adjustability of the height, and it needs to be measured several times during the surgery operation using multiple implant sizing tools with implants with a variation of sequentially increasing dimensions, wasting much of the operation time.

Adjustable spacers have also been developed. The problem with current adjustable spacers is that the spinal column is subject to extraordinary forces, even during normal physical activity. The spacers need to be able to withstand these extraordinary forces without failure or collapse. Due to these forces, the adjustable spacers are more prone to failure or collapse over time than the non-adjustable spacers.

What is needed are spacers that are height (i.e., thickness) adjustable, but that are very stable and are not prone to failure or collapse over time.

### SUMMARY

A height-adjustable vertebral spacer is described. The spacer has a superior member and an inferior member. The superior member has a superior vertebral interface and an inferior nesting interface. The superior vertebral interface includes angled teeth, and the inferior nesting interface has one or more lateral walls with one or more rows of slits formed therein. The inferior member has an inferior vertebral interface and one or more lateral walls with one or more rows of ridges protruding inwardly from the one or more lateral walls. Each of the ridges has an angled bottom side and a flat upper side that is perpendicular to the lateral wall from which it protrudes. When the superior member is mated with the inferior member the one or more ridges of the inferior member are oriented to mate with the one or more slits of the superior member.

### BRIEF DESCRIPTION OF THE DRAWINGS

FIG. 1 is a perspective view of a height-adjustable spacer.

FIG. 2 is a side view of the height-adjustable spacer depicted in FIG. 1.

FIG. 3 is a front view of the height-adjustable spacer depicted in FIG. 1.

FIG. 4 is a side cut-out view taken along lines A-A of FIG. 3.

FIG. 4a is a side cut-out view taken along lines B-B of FIG. 4, depicting the angle of the zip-lock teeth.

FIG. 5 is a perspective view of the height-adjustable spacer of FIG. 1 with the height adjusted to its shortest height.

FIG. 6 is a side view of the height-adjustable spacer depicted in FIG. 5.

FIG. 7 is a front view of the height-adjustable spacer depicted in FIG. 5.

FIG. 8 is a side cut-out view taken along lines A-A of FIG. 7.

FIG. 9A is top perspective view of a height-adjustable spacer in accordance with another embodiment.

FIG. 9B is a bottom perspective view of the height-adjustable spacer depicted in FIG. 9A.

### DETAILED DESCRIPTION

Described herein are height-adjustable spacers that are structurally stable and not prone to collapse from the forces exerted on them in the spinal column. FIG. 1 depicts a height-adjustable spacer 100 in accordance with one embodiment. The spacer 100 is made up of two components: a superior member 110 and an inferior member 120. Superior member 110 has a hollow core 90 that forms a cavity and is open on both its top side and bottom side. Inferior member 120 also has a hollow core 95 that forms a cavity and is open on both its top side and its bottom side.

Superior member 110 is made of two sections: a superior vertebral interface 112, and an inferior nesting interface 114. The superior vertebral interface 112 has a number of angled ridges or teeth 115 that allows the superior vertebral interface 112 to cut into and form a tight bond with the bone in the vertebra that rests down against the superior vertebral interface 112. The teeth 115 prevent the height-adjustable spacer 100 from slipping out of the vertebral column over time. All of the teeth 115 can be angled in the same direction as shown in FIGS. 1-5, or they can be angled in different directions relative to one another. For example, the teeth nearest the anterior end 140 of the superior vertebral interface 112 can be directed toward the anterior direction (not shown) while the teeth nearest the posterior end 150 of the superior vertebral interface 112 can be directed toward the posterior direction (as shown). Likewise, the opposite configuration can also be used with the teeth 115 nearest the anterior direction being directed toward the posterior direction (as shown) while the teeth 115 nearest the posterior end being directed toward the anterior direction (not shown).

Superior vertebral interface surface 112 has two separate and parallel rows of teeth 115 separated by hollow core 90. A first row 160a of teeth 115 runs along the length of superior vertebral interface surface 112 at one side of hollow core 90, and a second row 160b of teeth 115 runs along the length of superior vertebral interface surface 112 at the other side of hollow core 90. In FIGS. 1 and 5, teeth 115 in row 160a are angled in the same direction as teeth 115 in row 160b, in the posterior direction. In another embodiment, teeth 115 in row 160a and 160b can all be angled in the anterior direction instead of the posterior direction. In another embodiment as shown in FIG. 9, teeth 115 in row 160a are angled in the posterior direction while teeth 115 in row 160b are angled in the anterior direction. In yet another embodiment (not shown), teeth 115 in row 160a are angled in the anterior direction while teeth 115 in row 160b are angled in the posterior direction. The benefit of having teeth 115 of row 160a angled in the opposite direction as teeth 115 of row 160b is that once teeth 115 cut into the bone of the vertebrae, cage 100 will be prevented from slipping in either the anterior or posterior direction. Teeth 115 that are angled in the posterior direction will prevent cage 100 from slipping in a posterior direction, while teeth 115 that are angled in the anterior direction will prevent cage 100 from slipping in an anterior direction. Thus, cage 100 will not be able to slip in either direction once implanted.

Inferior nesting interface 114 has two lateral walls along its length that face each other, a posterior wall, and a narrowed anterior section. The lateral walls each have two columns of horizontal slits 118 (these can also be grooves instead of slits). The two columns of slits 118 on one lateral wall face and are opposite to the two columns of slits 118 on the opposing lateral wall, as can be seen in FIGS. 1 and 5.

Superior member 110 is hollow so that bone growth material can be inserted inside the hollow core and vertebral bone can grow and form through the hollow core. In this way, the successive vertebrae that are separated by the spacer 100 can fuse with one another.

Inferior member 120 has an open top end that is shaped to receive the inferior nesting interface 114 of the superior member 110. It also has an inferior vertebral interface 122 at its bottom side that allows it to interface with the bone of the vertebra on which it rests. The inferior vertebral interface 122 has a number of angled ridges or teeth 125 that allows the inferior vertebral interface 122 to cut into and form a tight bond with the bone in the vertebra that it rests atop. The teeth 125 prevent the height-adjustable spacer 100 from slipping out of the vertebral column over time. All of the teeth 125 can be angled in the same direction as shown in FIGS. 1-5, or they can be angled in different directions relative to one another. For example, the teeth nearest the anterior end 141 of the inferior member 120 can be directed toward the anterior direction (not shown) while the teeth nearest the posterior end 151 of the inferior member 120 can be directed toward the posterior direction (as shown). Likewise, the opposite configuration can also be used with the teeth 125 nearest the anterior direction being directed toward the posterior direction (as shown) while the teeth 125 nearest the posterior end being directed toward the anterior direction (not shown).

Like superior vertebral interface 112, inferior vertebral interface surface 122 has two separate and parallel rows of teeth 125 separated by hollow core 95. A first row 165a of teeth 125 runs along the length of inferior vertebral interface surface 122 at one side of hollow core 95, and a second row 165b of teeth 125 runs along the length of inferior vertebral interface surface 122 at the other side of hollow core 95. Teeth 125 in row 165a can be angled in the same direction as teeth 125 in row 165b, in the posterior direction. In another embodiment, teeth 125 in row 165a and 165b can all be angled in the anterior direction instead of the posterior direction. In another embodiment, teeth 125 in row 165a are angled in the posterior direction while teeth 125 in row 165b are angled in the anterior direction. In yet another embodiment, teeth 125 in row 165a are angled in the anterior direction while teeth 125 in row 165b are angled in the posterior direction. The benefit of having teeth 125 of row 165a angled in the opposite direction as teeth 125 of row 165b is that once teeth 125 cut into the bone of the vertebrae, cage 100 will be prevented from slipping in either the anterior or posterior direction. Teeth 125 that are angled in the posterior direction will prevent cage 100 from slipping in a posterior direction, while teeth 125 that are angled in the anterior direction will prevent cage 100 from slipping in an anterior direction. Thus, cage 100 will not be able to slip in either direction once implanted.

The angles of teeth 125 of inferior vertebral interface 122 can match and be directed in the same direction as corresponding teeth 115 on superior vertebral interface 112, i.e., teeth 115 of row 160a can be angled in the same direction as teeth 125 of row 165a, while teeth 115 of row 160b can be angled in the same direction as teeth 125 of row 165b. In one embodiment, teeth 115 of row 160a and teeth 125 of row 165a are angled in the posterior direction while teeth 115 of row 160b and teeth 125 of row 165b are angled in the anterior direction. In another embodiment, teeth 115 of row 160a and teeth 125 of row 165a are angled in the anterior direction while teeth 115 of row 160b and teeth 125 of row 165b are angled in the posterior direction.

In another embodiment as shown in FIGS. 9a and 9b, the angles of teeth 125 of inferior vertebral interface 112 can be in the opposite direction as those of corresponding teeth 115 on superior vertebral interface 112, i.e. teeth 115 of row 160a can be angled in the opposite direction as teeth 125 of row 165a, while teeth 115 of row 160b can be angled in the opposite direction as teeth 125 of row 165b. In one embodiment, teeth 115 of row 160a are angled in the posterior direction while teeth 125 of row 165a are angled in the anterior direction, and teeth 115 of row 160b are angled in the anterior direction while teeth 125 of row 165b are angled in the posterior direction. In another embodiment, teeth 115 of row 160a are angled in the anterior direction while teeth 125 of row 165a are angled in the posterior direction, and teeth 115 of row 160b are angled in the posterior direction while teeth 125 of row 165b are angled in the anterior direction. This configuration in which teeth 115 of rows 160a and 160b are angled in opposite directions and teeth 125 of rows 165a and 165b are the reverse of their corresponding rows 160a and 160b respectively, provides a uniquely stable mating between cage 100 and the vertebrae, preventing slippage in any direction and providing maximum stability once the cage is implanted.

Like superior member 110, inferior member 120 is also hollow and is open from its bottom side to its top side. Thus, when the superior and inferior members are mated with one another the assembled device is open on its top and bottom, allowing for bone to grow through the hollow center. In this way, the successive vertebrae that are separate by the spacer 100 can fuse with one another.

Inferior member 120 has two lateral walls along its length that face each other, a posterior wall 151, and a narrowed anterior section 141. The inner sides of the lateral walls each have two columns of horizontal ridges 121 that protrude inwardly toward the center of the hollow opening from the lateral walls. The two columns of ridges 121 on one of the lateral walls face and are opposite to the two columns of ridges 121 on the opposing lateral wall. As shown in FIG. 4a, each of the ridges has an upper side 121a that is flat or perpendicular to the lateral wall from which the ridge 121 protrudes, and a bottom side 121b that forms an obtuse angle with the lateral wall from which it protrudes. The ridges 121 are sized and positioned on the lateral walls to mate with the slits 118 on the lateral walls of the nesting interface 114 of the superior member 110 when the superior member 110 is inserted into the inferior member 120.

The spacer 100 can be inserted in between the vertebral body in its fully collapsed state, as shown in FIGS. 5-8. In the fully collapsed state, the ridges 121 of the inferior member 120 are mated with the slits 118 of the superior member 110. In one embodiment, the number of ridges 121 matches the number of slits 118. In another embodiment, the number of slits 118 exceeds the number of ridges 121. The superior member 110 can have 1, 2, 3, 4, 5, 6, 7, 8, 9, 10 or more rows of slits 118 in each of its columns of slits 118. The inferior member 120 can have 1, 2, 3, 4, 5, 6, 7, 8, 9, 10 or more (but not more than the number of rows of slits in its corresponding column of slits) rows of ridges 121 in each of its column of ridges 121.

Once the appropriate height for spacer 100 is determined, the superior member 110 and inferior member 120 can be forced apart, thus increasing the height of the spacer 100. As they are forced apart, superior member 110 slides upward, and the angled bottom side 121b of the ridge 121 allows for the slits 118 to slide over the ridge 121. Thus, expanding spacer 100 does not require undue force. However, once spacer 100 has been expanded, it cannot be collapsed by forces that squeeze the superior and inferior members together, because the slits cannot slide back over the flat upper side 121a of the ridges 121. This configuration of ridges 121 mating with slits 118 allows spacer 100 to expand, but it does not allow spacer 100 to collapse from forces that squeeze or push the superior and inferior members 110 and 120 toward one another. Spacer 100 cannot be collapsed without a compression tool that pulls the side walls of inferior member 120 away from one another while pushing superior member 110 down into inferior member 120. Thus, once spacer 100 is expanded it cannot be collapsed without a compression tool.

In another embodiment (not shown in the figures), ridges 121 on the inner sides of the lateral walls of inferior member 120 can be replaced with slits or grooves, while the slits or grooves 118 of the superior member are replaced with ridges, thus reversing the role of the two components when they mate. In such an embodiment, the ridges would have the opposite angling and orientation to the ridges 121 shown in FIG. 4a. The ridges would protrude outwardly from the outer wall of superior member 110 and would mate with the slits or grooves on the inner sides of the lateral walls of inferior member 120. The ridges have an upper side and a lower side, except they are oriented in the opposite direction as the ridges shown in FIG. 4a. The upper side of the ridges are angled downward and form an obtuse angle with the lateral outer wall of member 110, while the lower or bottom side of the ridges is flat or perpendicular to the lateral outer wall of member 110. In this configuration, the bottom side of the ridges, when mated with the slits or grooves, will rest against the shelves of the slits or grooves and will not be locked against the shelves of the slits or grooves. This will prevent member 110 and 120 to be compressed, i.e. superior member 110 collapsed into interior member 120 by compression forces alone. Thus, in this configuration, as in the one discussed above, spacer 100 cannot be collapsed without a compression tool that pulls the side walls of inferior member 120 away from one another while pushing superior member 110 down into inferior member 120. Thus, once spacer 100 is expanded it cannot be collapsed without a compression tool.

The spacer 100 can be made of any medical grade implantable material, such as stainless steel, medical grade plastic, titanium or titanium alloys, polyetheretherketone (PEEK), reinforced plastic, and pyrolitic carbon, including pyrolitic carbon able to receive an electrical signal. If the material is pyrolotic carbon able to receive an electrical signal, the spacer can be activated to stimulate bone growth by receiving an external or remote electrical signal. The electrically activated spacer will promote bone growth.

The spacer 100 is shaped like a boat with a narrower anterior end 114/141 than its posterior end 150/151. The reason for that is that the pointed anterior end makes it easier to implant the spacer in the posterior to anterior direction, which is the preferred implant method. However, the spacer can take other shapes, such as square, rectangular, round, oval, almond shaped, concave, convex, or hour-glass shaped.

While the invention is susceptible to various modifications and alternative forms, specific examples thereof have been shown by way of example in the drawings and are herein described in detail. It should be understood, however, that the invention is not to be limited to the particular forms or methods disclosed, but to the contrary, the invention is to cover all modifications, equivalents, and alternatives falling within the spirit and scope of the appended claims.

I claim:

1. A height-adjustable vertebral spacer comprising:
a superior member comprising a superior vertebral interface and an inferior nesting interface, wherein the superior vertebral interface comprises angled teeth, and the inferior nesting interface comprises one or more lateral walls with one or more rows of slits formed therein;
an inferior member comprising an inferior vertebral interface and one or more lateral walls with one or more rows of ridges protruding inwardly from the one or more lateral walls, wherein each of the ridges has an angled bottom side and a flat upper side that is perpendicular to the lateral wall from which it protrudes, and wherein the inferior vertebral interface comprises angled teeth;
wherein when the superior member is mated with the inferior member, the one or more ridges of the inferior member are oriented to mate with the one or more slits of the superior member.

2. The vertical spacer of claim 1, wherein an anterior end of the spacer is narrower than the posterior end.

3. The vertical spacer of claim 1, wherein the spacer can be expanded in height without a compression tool but cannot be compressed without a compression tool.

4. The vertical spacer of claim 1, wherein the angled teeth of the superior vertebral interface are oriented in the same direction as the angled teeth of the inferior vertebral interface.

5. The vertical spacer of claim 1, wherein the angled teeth of the superior vertebral interface are oriented in the opposite direction as the angled teeth of the inferior vertebral interface.

6. The vertical spacer of claim 4, wherein the angled teeth of the superior vertebral interface and the angled teeth of the inferior vertebral interface are all angled toward the anterior direction.

7. The vertical spacer of claim 4, wherein the angled teeth of the superior vertebral interface and the angled teeth of the inferior vertebral interface are all angled toward the posterior direction.

8. The vertical spacer of claim 1, wherein the superior member comprises a hollow space and wherein the superior vertebral interface comprises a first row of teeth on one side of the hollow space and a second row of teeth on the other side of the hollow space.

9. The vertical spacer of claim 8, wherein the first row of teeth are angled in the same direction as the second row of teeth.

10. The vertical spacer of claim 8, wherein the first row of teeth are angled in the opposite direction as the second row of teeth.

11. The vertical spacer of claim 8, wherein the inferior member comprises a hollow space and wherein the inferior vertebral interface comprises a first row of teeth on one side of the hollow space and a second row of teeth on the other side of the hollow space.

12. The vertical spacer of claim 11, wherein the first row of teeth are angled in the same direction as the second row of teeth.

13. The vertical spacer of claim 11, wherein the first row of teeth are angled in the opposite direction as the second row of teeth.

14. The vertical spacer of claim 13, wherein the first row of teeth of the superior member and the corresponding first row of teeth in the inferior member are angled in the opposite direction from one another, and the second row of teeth of the superior member and the corresponding second row of teeth in the inferior member are angled in the opposite direction from one another.
